(19) United States
(12) Patent Application Publication
(10) Pub. No.: US 2009/0299373 A1
(43) Pub. Date: Dec. 3, 2009

(54) KYPHOPLASTY BANDED BALLOON CATHETER

(75) Inventor: Richard B. Sisken, West Lafayette, IN (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(73) Assignee: COOK INCORPORATED, Bloomington, IN (US)

(21) Appl. No.: 12/130,531

(22) Filed: May 30, 2008

(51) Int. Cl. A61B 17/58; A61M 29/00

(52) U.S. Cl. 606/92; 606/192

(57) ABSTRACT

A balloon catheter for insertion into a vertebral body is provided. The balloon catheter includes a shaft having a proximal end and a distal end. A lumen extends through the shaft along a longitudinal axis. An inflatable balloon having an uninflated configuration and an inflated configuration is disposed near the distal end of the shaft. The balloon has a proximal portion and a distal portion and an interior and an exterior. The lumen is in fluid communication with the interior of the balloon. An expandable band is wrapped around a section of the exterior of the balloon in a generally proximal-distal direction. The balloon catheter is configured to be positioned within the vertebral body such that the expandable band is configured to prevent leakage of a bone-filling material in a generally lateral direction relative to a vertebral body and facilitate adhesion of the bone-filling material within the vertebral body.

### BACKGROUND

1. Field of the Invention

The present invention generally relates to kyphoplasty. More particularly, the invention relates to a method and apparatus for performing kyphoplasty.

2. Description of Related Art

Vertebral compression fractures represent a generally common spinal injury, cause pain and spinal deformity, and may result in prolonged disability. These fractures involve the collapse of one or more vertebral bodies in the spine and can result, for example, from metastatic diseases of the spine, from trauma, or can be associated with osteoporosis.

Kyphoplasty is a minimally-invasive procedure that has been developed to access and treat diseased or fractured bone, such as collapsed or fractured vertebral bodies. In a kyphoplasty procedure, a surgeon manipulates the cancellous and/or cortical bone of the weakened and/or fractured vertebral body with surgical tools, and then introduces a bone-filling material such as bone cement into the bone, desirably into a cavity formed within the vertebral body, in an attempt to repair, reinforce and/or prevent further fracture or subsidence of the bone. This procedure seeks to reduce the pain and discomfort experienced by patients suffering from vertebral compression fractures.

A source of complication with the kyphoplasty technique is leakage of the bone-filling material which can cause compression of the spine or the dorsal nerves, or pulmonary embolism caused by leakage of the bone-filling material into the veins. Because of this hazard, the bone-filling material must be very viscous. This requires large needles, high needle insertion force, and high injection pressure, all of which create risk to the patient. Leakage into the arterial system is less of a concern because the arteries break up into small arterioles. Venous returns, however, are located in the center of the anterior and posterior walls of the vertebral cavity and are large. Therefore, leakage of the bone-filling material into the veins is a major concern.

To lessen leakage complications, a kyphoplasty procedure using a resorbable balloon-tipped catheter has been proposed, wherein the catheter is inserted into a fractured vertebral body and a bone-filling material is injected through the catheter and into the expandable balloon to stabilize the fracture. The balloon is held in place until the bone-filling material begins to set which prevents leakage of the bone-filling material into the bloodstream. The catheter is removed and the balloon remains in place within the vertebral body and resorbs over time. This technique minimizes the opportunities for leakage of the bone-filling material outside of the targeted bone.

While this procedure has been shown to reduce some pain associated with vertebral compression fractures, it has certain inherent drawbacks. The bone-filling material is contained within the balloon as it sets, preventing adhesion of the bone-filling material to the vertebral fragments. Although the balloon filled with the bone-filling material may provide a large enough lump in between vertebral fragments to prevent them from grinding together, the bone-filling material is not well integrated with the vertebral body which can lead to relative motion between the bone-filling material and the vertebral fragments, causing the patient pain.

Accordingly, there is a need to provide an improved method and apparatus for performing kyphoplasty which relieves pain caused by vertebral compression fractures and prevents leakage of bone-filling materials into the bloodstream of a patient.

### SUMMARY

Embodiments of the present invention provide a balloon catheter and a method for performing kyphoplasty which promotes healing of fractured vertebral bodies and prevents leakage of a bone-filling material into the bloodstream of a patient, thereby reducing patient discomfort associated with vertebral fractures. This invention also allows the use of a less viscous bone-filling material, which improves working time and allows for smaller instruments (i.e., a smaller injection needle and balloon catheter).

In at least one embodiment of the present invention, a balloon catheter for insertion into a vertebral body having first and second endplates is provided. The balloon catheter comprises a shaft having a proximal end and a distal end. A lumen extends through the shaft along a longitudinal axis. An inflatable balloon is positioned near the distal end of the shaft. The balloon has an uninflated configuration and an inflated configuration. The balloon has a proximal portion and a distal portion, an interior and an exterior. The lumen is in fluid communication with the interior of the balloon. An expandable band is wrapped around a section of the exterior of the balloon in a generally proximal-distal direction.

In at least one other embodiment, a balloon catheter for insertion into a vertebral body having first and second endplates is provided is provided. The balloon catheter comprises a shaft having a proximal end and a distal end. A lumen extends through the shaft along a longitudinal axis. An inflatable balloon is positioned near the distal end of the shaft. The balloon has an uninflated configuration and an inflated configuration. The balloon has a proximal portion and a distal portion, an interior and an exterior. The lumen is in fluid communication with the interior of the balloon. An expandable band is wrapped around a section of the exterior of the balloon in a generally proximal-distal direction. The balloon includes a first section, a second section, and a third section. The second section is disposed between the first and third sections and the band is wrapped around the second section. The balloon catheter is configured to be inserted into the vertebral body such that the first section of the balloon substantially opposes the first endplate and the third section of the balloon substantially opposes the second endplate such that the band wrapped around the second section of the balloon is configured to expand in a generally lateral direction relative to the vertebral body when the balloon is inflated.

In at least one other embodiment, a method of performing kyphoplasty is provided. The method comprises providing a balloon catheter including a shaft having a distal end and a proximal end. A lumen extends through the shaft along a longitudinal axis. An inflatable balloon having an uninflated configuration and an inflated configuration is positioned near the distal end of the shaft. The balloon includes a proximal portion and a distal portion and an interior and an exterior. The lumen is in fluid communication with the interior of the balloon. An expandable band is wrapped around a section of the exterior of the balloon in a generally proximal-distal direction.

The balloon catheter is positioned for insertion into a vertebral body having first and second endplates and inserted into the vertebral body when the balloon is in the uninflated configuration. A cavity is formed within the vertebral body by inflating the balloon to the inflated configuration which expands the band to an expanded configuration. The balloon catheter is removed from the vertebral body by deflating the balloon to the uninflated configuration and removing the shaft and balloon from the vertebral body. The band is configured to at least partially maintain the expanded configuration during deflation of the balloon and remains within the vertebral body during removal of the shaft and the balloon. A bone-filling material is dispensed into the cavity of the vertebral body. The band is configured to prevent leakage of the bone-filling material in a generally lateral direction relative to the vertebral body and facilitate adhesion of the bone-filling material to the vertebral body in a generally vertical direction relative to the vertebral body.

Further objects, features and advantages of this invention will become readily apparent to persons skilled in the art after a review of the following description, with reference to the drawings and claims that are appended to and form a part of this specification.

### BRIEF DESCRIPTION OF THE DRAWINGS

FIG. 1 is a partial side view of a collapsed vertebra;

FIG. 2 is a partial side view of a collapsed vertebral body;

FIG. 3 is a top view of FIG. 2;

FIG. 4a is a side view of a device for stabilizing a collapsed vertebral body in accordance with an embodiment of the present invention, showing the balloon in the uninflated configuration and the band in the unexpanded configuration;

FIG. 4b is a top cross-sectional view of FIG. 4a taken along axis A;

FIG. 5a is a side view of the device in FIG. 4a, showing the balloon in the inflated configuration and the band in the expanded configuration;

FIG. 5b is a top cross-sectional view of FIG. 5a taken along axis A;

FIG. 6a is a side view of the device in FIG. 4a, showing the balloon in the uninflated configuration and the band in the expanded configuration;

FIG. 6b is a top cross-sectional view of FIG. 6a taken along axis A;

FIGS. 7a-d are partial side views of a device for stabilizing a collapsed vertebral body in accordance with embodiments of the present invention;

FIG. 7e is a top cross-sectional view of FIG. 7d;

FIG. 8a is a partial side view of a device for stabilizing a collapsed vertebral body in accordance with another embodiment of the present invention;

FIG. 8b is a partial side view of a stabilized collapsed vertebra in accordance with one example of the present invention;

FIG. 9a is a side view of a device for stabilizing a collapsed vertebral body in accordance with another embodiment of the present invention, showing the balloon in the uninflated configuration and the band in the unexpanded configuration;

FIG. 9b is a side view of a device for stabilizing a collapsed vertebral body in accordance with another embodiment of the present invention, showing the balloon in the uninflated configuration and the band in the unexpanded configuration; and

FIG. 10 is a flow chart for a method of performing kyphoplasty.

### DETAILED DESCRIPTION

The present invention generally provides a balloon catheter device for treating damaged vertebral bodies. The device forms a cavity within a damaged vertebral body for receiving a bone-filling material and prevents leakage of the bone-filling material into a patient's bloodstream. Embodiments of the present invention seek to overcome the complications associated with leakage of the bone-filling material and promote adhesion of the bone-filling material with vertebral fragments within the vertebral body to repair the damaged vertebral body and reduce patient discomfort.

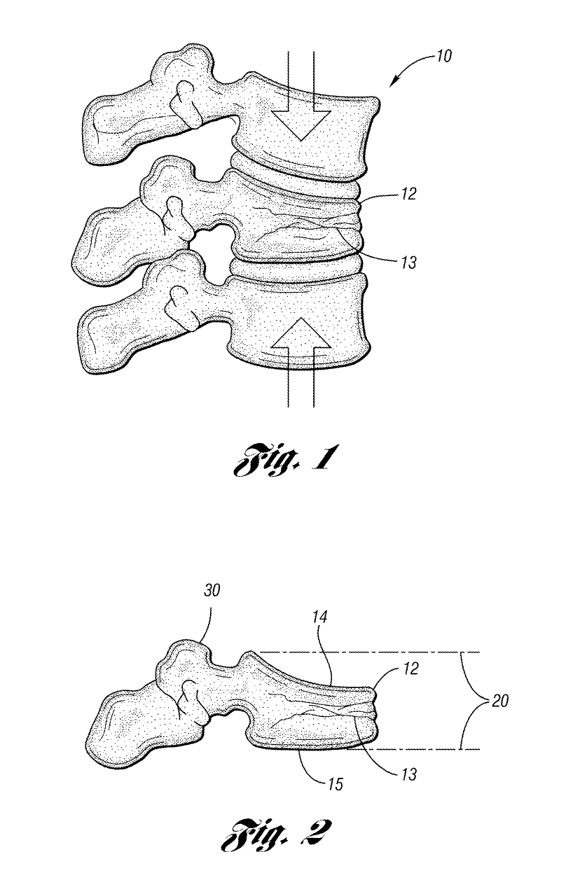

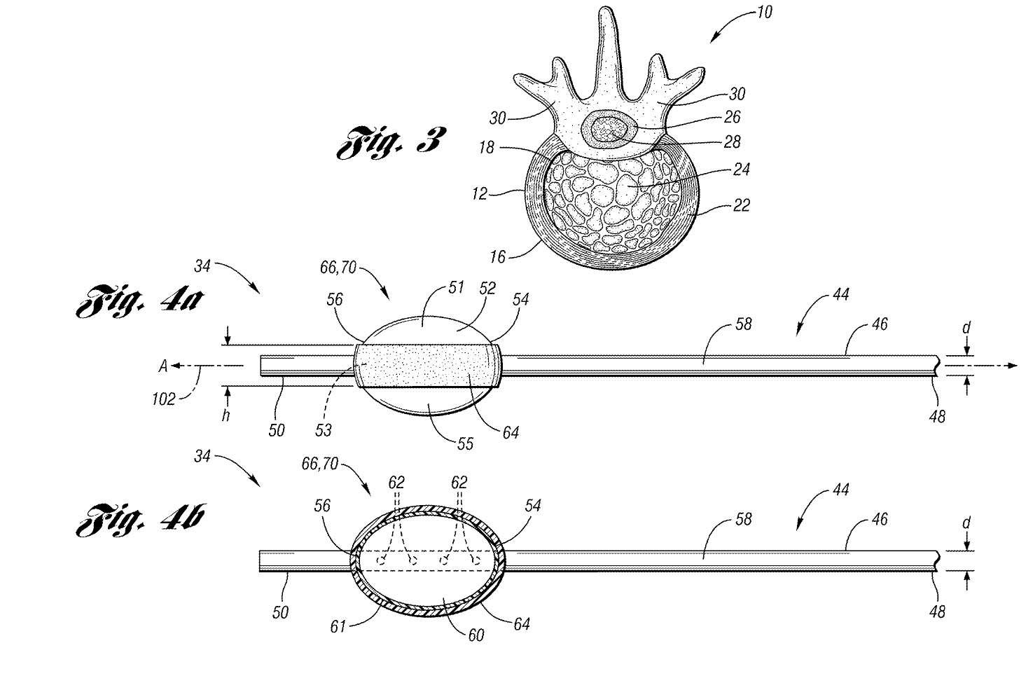

Referring now to the drawings, FIG. 1 illustrates a vertebra 10 which includes a collapsed vertebral body 12 with a compression facture 13. The vertebra 10 may be, for example, in the thoracic or lower spine of the patient. As depicted in FIG. 2, the vertebral body 12 has fractured, with the first and second endplates 14 and 15 depressing generally towards each other and away from their pre-fractured, normally parallel, orientation (indicated generally as parallel lines 20). This condition is commonly caused by osteoporosis or trauma to the back.

FIG. 3 depicts an enlarged top view of the vertebral body 12 of FIG. 2. The vertebral body 12 extends on the anterior (i.e., front or chest) side of the vertebra 10. The vertebral body 12 is approximately the shape of an oval disk, with an anterior wall 16 and a posterior wall 18. The geometry of the vertebral body 12 is generally symmetric. The vertebral body includes an exterior formed from compact cortical bone 22. The cortical bone 22 encloses an interior volume of reticulated cancellous, or spongy, bone 24. The spinal canal 26 is located on the posterior (i.e., back) side of each vertebra 10. The spinal cord 28 passes through the spinal canal 26. Left and right pedicles 30 adjoin the vertebral body 12.

The collapsed vertebral body 12 may be stabilized by kyphoplasty, a medical procedure for introducing a bone-filling material into the collapsed vertebra. This procedure stabilizes the collapsed vertebra by forming a cavity within the interior open spaces within the cancellous bone of the vertebral body 12 which is subsequently filled with a bone-filling material to provide a more continuous and solid form. Kyphoplasty may further stabilize the vertebral body 12 by restoring vertebral spacing which alleviates nerve pinching from the vertebra 10. It should be noted that the present invention applies to this medical procedure and other procedures for stabilizing and/or repairing damaged bones of patients.

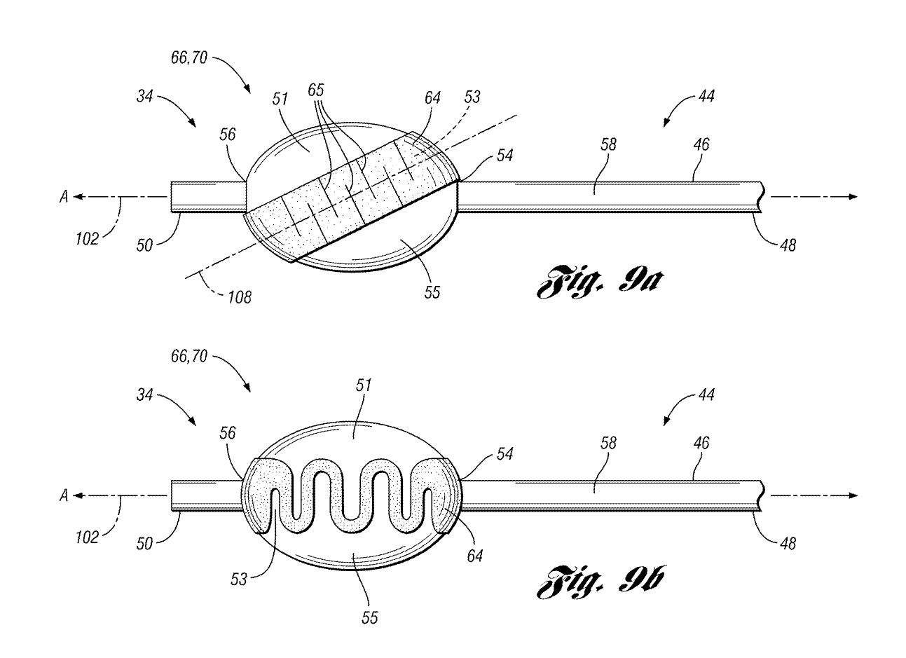

Desirably, kyphoplasty is performed under a local anesthesia and/or a light sedative. A small nick is made in the skin near the spine and an insertion instrument is inserted percutaneously into the fractured vertebral body 12 through, preferably, a targeted area of the patient's back. The insertion instrument may be any type and size of hollow instrument, preferably having a sharp end. For example, the insertion instrument may include a hollow needle having a hollow lumen configured to permit the passage of various instruments and materials. The hollow needle, for instance, may be anywhere from a six to a 14 gage needle. As illustrated in FIG. 8a, the needle 32 may be inserted into the cancellous bone of the vertebral body 12, for example via or through the left or right pedicle 30 of the vertebral body 12.

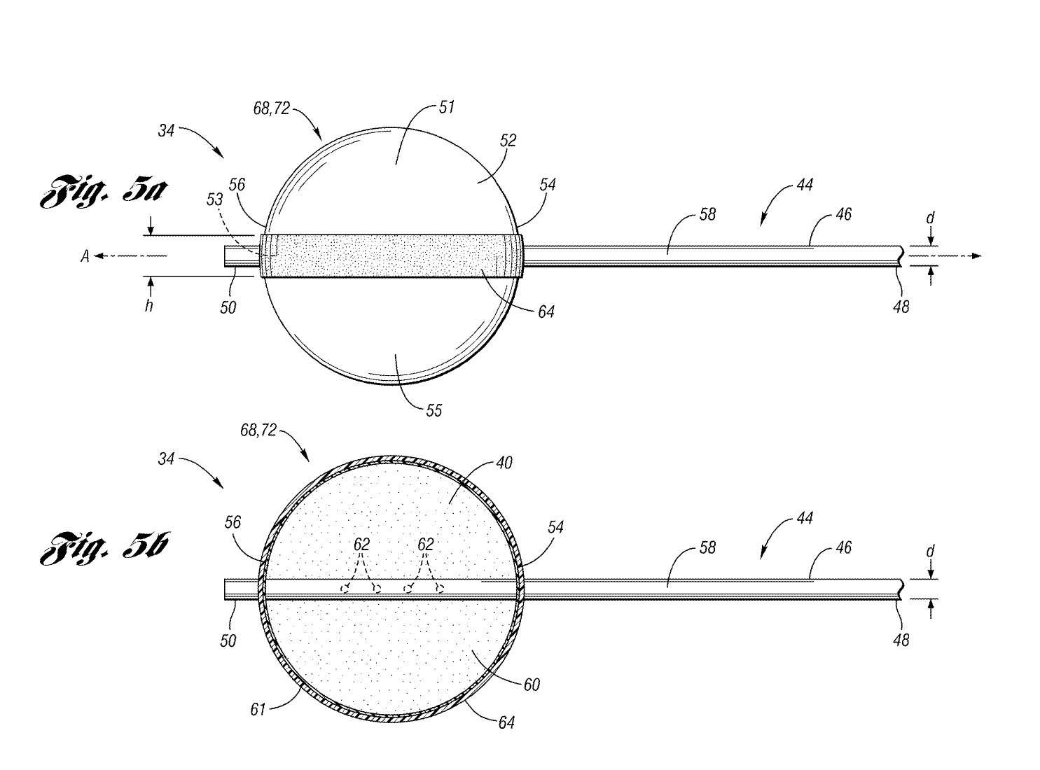

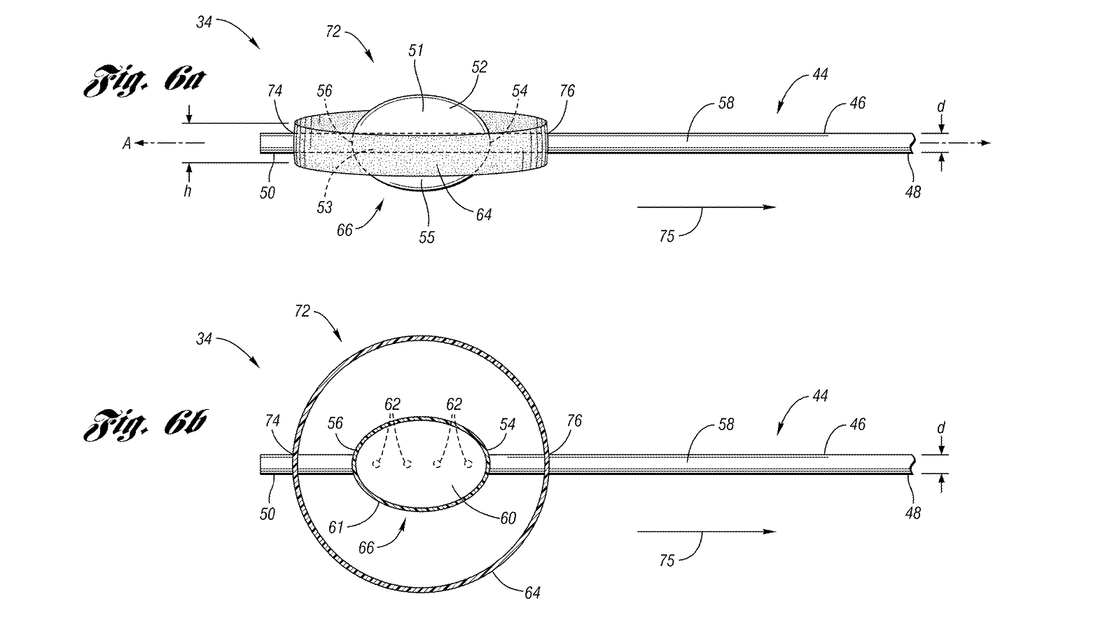

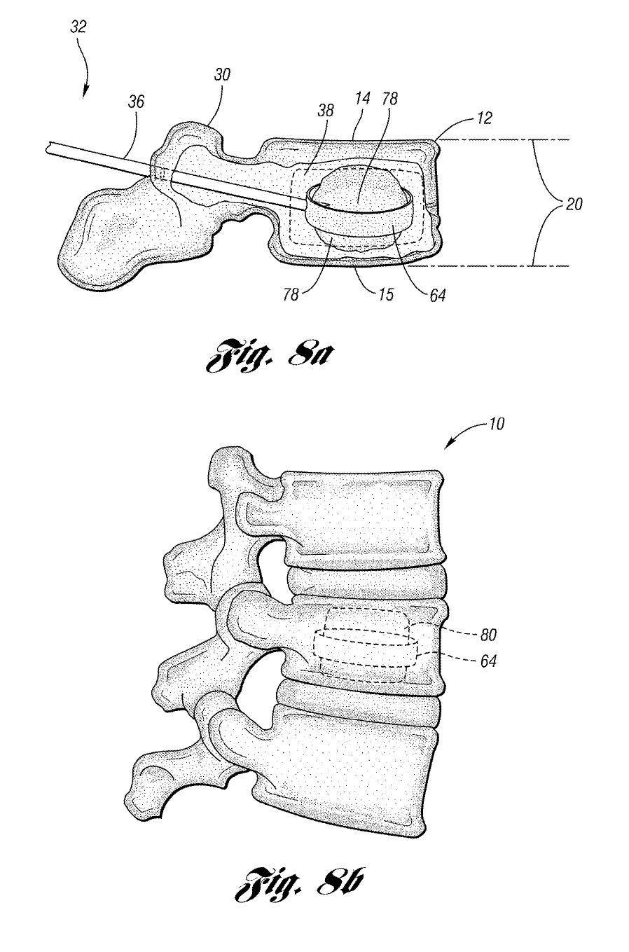

Referring to FIGS. 7b-e, once the needle 32 is in position, a balloon catheter 34 is inserted through the needle body 36 and into the vertebral body 12 to displace cancellous bone and thereby form a cavity 38 within the vertebral body 12. In a preferred embodiment, as illustrated in FIGS. 4a-6b, the balloon catheter 34 comprises a catheter 44 having a hollow shaft 46 including a proximal end 48 and a distal end 50. The shaft 46 is preferably formed of a plastic or rubber material, stainless steel, or any other suitable material. In this embodiment, an inflatable balloon 52, comprised of a flexible material such as commonly used for balloon catheters including, but not limited to, plastics, composite materials, polyethylene, mylar, rubber, polyurethane, or any other suitable material, is positioned near the distal end 50 of the shaft 46. In this embodiment, the distal end 50 of the shaft 46 pierces through the balloon 52, first piercing through a proximal portion 54 of the balloon 52 and then through a distal portion 56 of the balloon 52. A lumen 58 extends through the shaft 46 along a longitudinal axis A. The lumen 58 is in fluid communication with the interior 60 of the balloon 52 via at least one opening 62 formed through the shaft 46 near the distal end 50.

In this embodiment, an expandable, and preferably resorbable, band 64 is wrapped around a section of the balloon 52 in the proximal-distal direction, i.e., the band 64 is wrapped around the balloon 52 from at least part of the proximal portion 54 of the balloon 52 toward at least part of the distal portion 56 of the balloon 52. As illustrated in the embodiments of FIGS. 4a, 5a, 6a, and 9a-b, the balloon 52 can be described as having a first section 51, a second section 53, and a third section 55. The band 64 is wrapped around the second section 53 which is disposed between the first section 51 and the third section 55. The second section 53 may be substantially parallel to the longitudinal axis A (FIGS. 4a, 5a, 6a). Referring to FIGS. 4a, 5a, and 6a, the second section 53 may be coplanar with the longitudinal axis A, defining a plane 102 along which the top cross-sectional views of FIGS. 4b, 5b, and 6b are taken.

In this embodiment, the balloon catheter 34 is configured to fit through the hollow interior of the needle body 36 and into the vertebral body 12. An adhesive may be used to keep the band 64 in place around the balloon 52 as the balloon catheter 34 is pushed through the needle body 36 and introduced into the vertebral body 12. The balloon catheter 34 is configured to be positioned within the vertebral body 12 so that the first section 51 of the balloon 52 substantially opposes either of the first and second endplates 14, 15 and the third section 55 of the balloon 52 substantially opposes the other one of the first and second endplates 14, 15.

Thus, the balloon catheter 34 is configured to be positioned within the vertebral body 12 such that the band 64 wrapped around the second section 53 of the balloon 52 is substantially parallel to the first and second endplates 14, 15 and configured to expand generally laterally with respect to the vertebral body 12 when the balloon is inflated. The balloon catheter 34 is likely inserted into the vertebral body 12 on an angle. Therefore, the substantially parallel description includes a parallel relationship, as well as a slightly angled relationship, between the first, second, and third sections 51, 53, and 55 of the balloon 52 and the first and second endplates 14, 15 such that the band 64 wrapped around the second section 53 of the balloon 52 is configured to expand generally laterally with respect to the vertebral body 12.

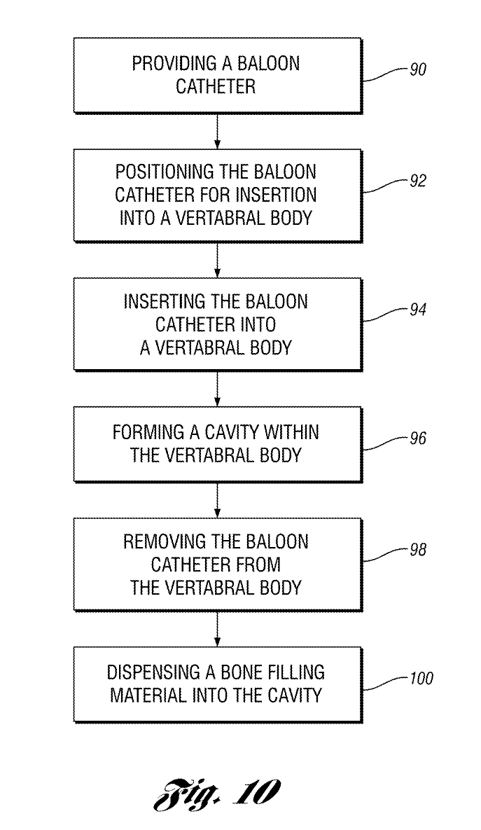

Referring to FIG. 9a, the second section 53 may be defined by a plane 108 which is tilted from the longitudinal axis A and intersects the plane 102. In other words, the second section 53, and thus the band 64, may be aparallel to the longitudinal axis A. This embodiment takes into account the situation where the balloon catheter 34 is likely inserted into the vertebral body 12 on an angle. The positioning of the band 64 along a plane 108 aparallel to the longitudinal axis A and in the proximal-distal direction (i.e., wrapped around the balloon 52 from at least a part of the proximal portion 54 toward at least a part of the distal portion 56) allows the band 64 to expand in a generally lateral direction with respect to the vertebral body 12. In this embodiment, the shaft 46 does not pierce through the band 64. As illustrated in FIG. 9a, the band 64 may include a plurality of vertical slits 65 disposed on the band 64 to enhance the expandability of the band 64.

In the embodiments illustrated in FIGS. 4a, 5a, 6a, and 9a, the band 64 has a generally uniform height (h). Referring to FIGS. 4a, 5a, and 6a, the height (h) is greater than the diameter (d) of the shaft 46 such that the distal end 50 of the shaft 46 pierces through the band 64 before it pierces through the balloon 52 at the proximal portion 54 and after it pierces through the balloon 52 at the distal portion 56. In another embodiment, the band 64 may have a non-uniform height (h). As illustrated in FIG. 9b, for example, the band 64 may have portions having a height greater than the diameter (d) of the shaft 46 and portions having a height less than the diameter (d) of the shaft 46.

During insertion of the balloon catheter 34, the location of the balloon catheter 34 may be monitored using visualization equipment such as real-time X-Ray, CT scanning equipment, MRI, or any other commonly used monitoring equipment. In this embodiment, once in a desired position within the vertebral body 12, the lumen 58 supplies the balloon 52 with an appropriate volume of inflation medium 40 suitable for use in medical applications including, but not limited to, air, nitrogen, saline or water. The inflation medium 40 may further include a radiopaque fluid to allow the physician to visualize the balloon catheter 34 during inflation. As the inflation medium 40 fills the balloon 52, the balloon 52 inflates from the uninflated configuration 66 to the inflated configuration 68. Likewise, the band 64 expands with the balloon 52 from an unexpanded configuration 70 to an expanded configuration 72.

As the balloon 52 inflates the adhesive bond between the balloon 52 and the band 64 breaks. Alternatively, the hydration of a film of suitable material between the balloon 52 and the band 64, used to hold the balloon 52 and the band 64 together, may cause the balloon 52 and band 64 to become uncoupled. Suitable materials for this application may include many types of water-based gels that become solid when desiccated. With the inflation of the balloon 52, the first section 51 of the balloon 52 moves generally toward the first endplate 14 and the third section 55 of the balloon 52 moves generally toward the second endplate 15, thereby displacing cancellous bone and forming a cavity 38 within the vertebral body 12.

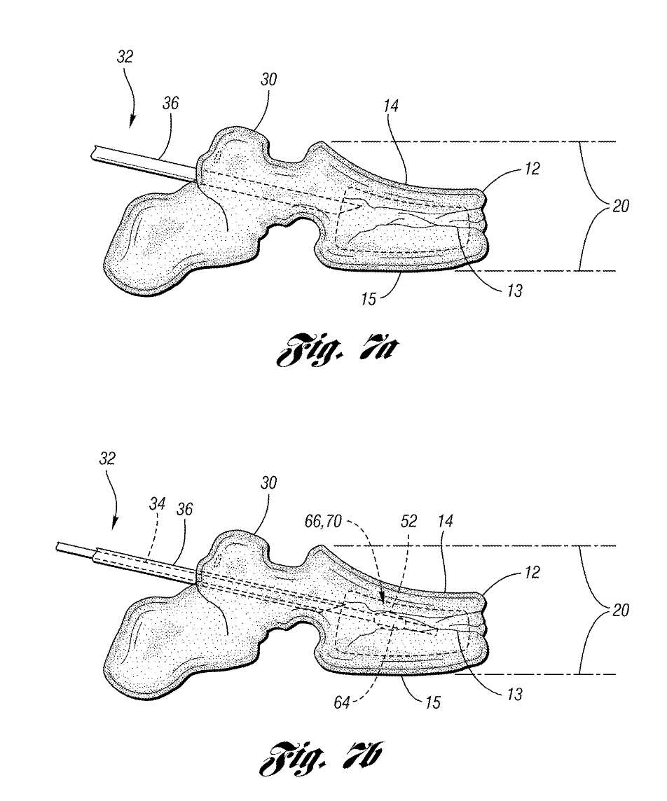

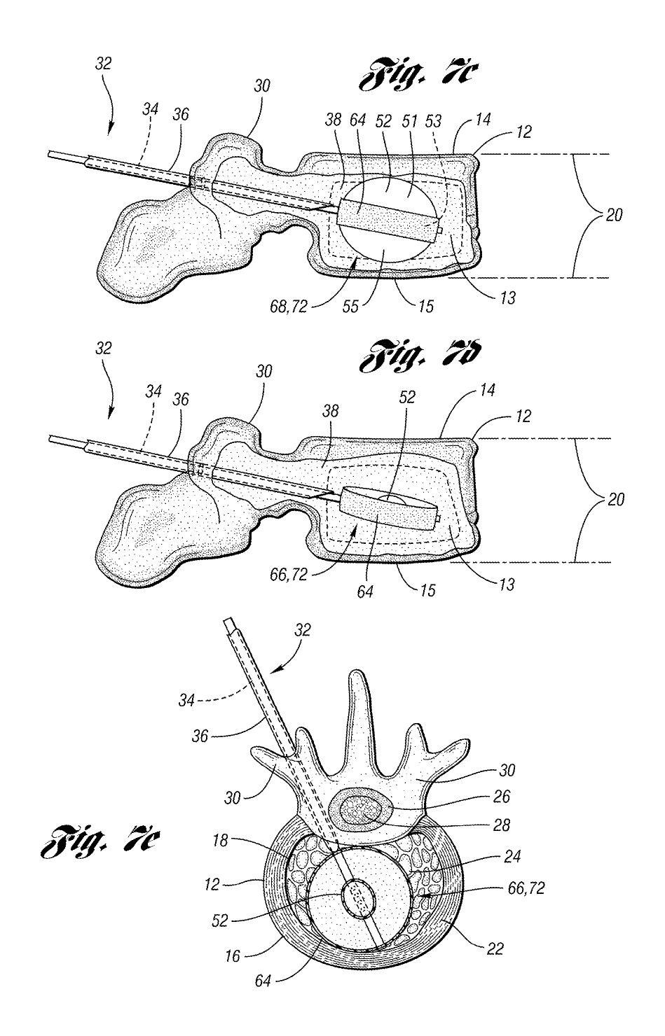

In this embodiment, once a desired cavity size is reached and the inflated balloon 52 has provided sufficient pressure to force at least one of the first and second endplates 14 and 15 of the vertebral body 12 back into their pre-fractured configurations (FIG. 7c), the inflation medium 40 is withdrawn from the balloon catheter 34 to deflate the balloon 52 to the uninflated configuration 66 (FIGS. 7d-e), and the balloon catheter 34 is removed from the vertebral body 12 and the needle body 36. During deflation of the balloon 52 and removal of the balloon catheter 34, the band 64 is configured to at least partially maintain its expanded configuration 72. In this embodiment, as the balloon catheter 34 is removed, the distal end 50 of the shaft 46 is withdrawn through an opening in the distal portion 74 of the band 64. Subsequently, the uninflated balloon 52 and the distal end 50 are withdrawn through an opening in the proximal portion 76 of the band 64. The shaft 46 is retracted in the direction of arrow 75 (FIGS. 6a-b), through the opening in the proximal portion 76 of the band 64.

The band 64 is preferably formed of small intestinal submucosa (SIS). SIS is a resorbable, acellular, naturally occurring tissue matrix composed of extracellular matrix (ECM) proteins in various growth factors. SIS is derived from the porcine jejunum and functions as a remolding bioscaffold for tissue repair. SIS has characteristics of an ideal tissue engineered biomaterial and can act as a bioscaffold for remodeling of many body tissues including skin, body wall, musculoskeletal structure, urinary bladder, and also supports new blood vessel growth. SIS has been shown to be completely replaced by the patient's own tissues over time. Alternatively, the band 64 may be formed of any other suitable implantable material including, but not limited to, polylactic acid, polyglycolic acid, poly (ortho esters), poly (glycolide-co-trimethylene carbonate), poly-L-lactide-co-6-coprolactone, polyanhydrides, poly-n-dioxanone, poly (PHB-hydroxyvaleric) acid, surgical mesh, graft fabric, and any mixture or composite thereof.

Referring to FIG. 8a, after removal of the balloon catheter 34 from the vertebral body 12 and the needle 32, a bone-filling material 78 is dispensed from a device (not shown) through the needle 32 and into the vertebral body 12 to form a solid structure 80 that stabilizes the collapsed vertebral body 12. The bone-filling material 78 could be any appropriate filling material used in orthopedic surgery, including, but not limited to, PMMA bone cement, allograft or autograft tissue, hydroxyapatite epoxy, or synthetic bone substitutes. In this embodiment, as shown in FIG. 8a, the needle 32 moves forward through the opening in the distal portion 74 of the band 64 before injecting the bone-filling material 78 into the cavity formed by the balloon catheter within the vertebral body 12. The positioning of the band 64 is configured to substantially prevent leakage of the bone-filling material 78 in generally the lateral direction relative to the vertebral body 12, yet it is not configured to prevent leakage of the bone-filling material 78 in generally the vertical direction relative to the vertebral body 12. Thus, the band 64 is configured to prevent the bone-filling material 78 from entering the bloodstream of the patient (generally laterally) and to allow the bone-filling material 78 to adhere to the first and second endplates 14 and 15 of the vertebral body 12 (generally vertically) before forming a solid structure 80 by chemically reacting or curing to become a solid (FIG. 8b).

As opposed to solutions which seek to prevent leakage of the bone-filling material in all directions, by injecting the bone-filling material into a resorbable balloon and allowing it to cure within the balloon, the band 64, in accordance with embodiments of the present invention, promotes integration of the bone-filling material 78 within the vertebral body 12 and substantially reduces pain associated with the grinding between the solidified bone-filling material 78 and vertebral fragments. Further, the stabilizing solid structure 80 helps restore vertebral spacing and alleviate nerve pinching by adhering to and supporting the collapsed vertebral body 12. Preferably, the structure 80 generally fills in the open spaces of the collapsed vertebral body 12 providing a more dense and continuous vertebral body 12 which enhances mobility of the patient. The band 64, preferably made of SIS, remains in the vertebral body 12 and promotes tissue growth. The positioning of the band 64 allows the body cells to adhere to the SIS and subsequently differentiate, growing into the SIS. The ability to block the venous return from the vertebral body 12 allows the bone-filling material 78 to be less viscous and the instruments (i.e., the needle 32 and balloon catheter 34, as well as the injection system (not shown)) to be smaller and operate under lower pressures. A less viscous bone-filling material 78 will also allow a longer working time of the material, so that the clinician is not as rushed.

It is also within the scope of the present invention for the balloon 52 to be attached to an opening at the distal end 50 of the shaft 46 such that the shaft 46 does not pierce through the balloon 52, but rather the proximal portion 54 of the balloon is bonded to the distal end 50 of the shaft 46 by means known in the art, such as by a suitable adhesive. In this embodiment, the lumen 58 and the interior 60 of the balloon 52 may be in fluid communication via an opening at the distal end 50 of the shaft 46. In this embodiment, the band 64 may be at least partially wrapped around the second section 53 of the balloon 52 and may be pierced by the distal end 50 of the shaft 46 at the proximal portion 76 of the band 64 so as not to obstruct the fluid communication between the lumen 58 and the interior 60 of the balloon 52. When the balloon catheter 34 is removed from the vertebral body 12 after inflation and deflation of the balloon 52 and expansion of the band 64, the distal end 50 of the shaft 46, including the deflated balloon 52, may be withdrawn through an opening in the proximal portion 76 of the band 64 before being withdrawn through the needle 32.

Referring to FIG. 10, a method of performing kyphoplasty is provided. The method includes providing a balloon catheter (90) as discussed in the forgoing paragraphs.

The balloon catheter is positioned for insertion into a vertebral body (92). The balloon catheter is then inserted into the vertebral body (94).

A cavity is formed within the vertebral body (96). This includes inflating a balloon to an inflated configuration. This causes a band wrapped around a section of the balloon to expand to an expanded configuration.

The balloon catheter is removed from the vertebral body (98). This includes deflating the balloon to an uninflated configuration and removing a shaft and the balloon of the balloon catheter from the vertebral body. The band is configured to at least partially maintain the expanded configuration during deflation of the balloon. The band remains within the vertebral body during removal of the shaft and the balloon.

A bone-filling material is dispensed into the cavity of the vertebral body (100). The band prevents leakage of the bone-filling material in generally a lateral direction relative to the vertebral body and facilitates adhesion of the bone-filling material to the vertebral body in generally a vertical direction relative to the vertebral body.

As a person skilled in the art will readily appreciate, the above description is meant as an illustration of implementation of the principles this invention. This description is not intended to limit the scope or application of this invention in that the invention is susceptible to modification, variation and change, without departing from spirit of this invention, as defined in the following claims.

1. A balloon catheter for insertion into a vertebral body having first and second endplates, the balloon catheter comprising:
a shaft having a proximal end, a distal end, and a lumen extending therethrough along a longitudinal axis;
an inflatable balloon having an uninflated configuration and an inflated configuration disposed near the distal end of the shaft, the balloon having a proximal portion and a distal portion and an interior and an exterior, the interior being in fluid communication with the lumen; and
an expandable band wrapped around a section of the exterior of the balloon in a generally proximal-distal direction.

2. The balloon catheter of claim 1 wherein the balloon includes a first section, a second section, and a third section, the second section disposed between the first section and the third section, the band being wrapped around the second section.

3. The balloon catheter of claim 2 wherein the balloon catheter is configured to be inserted into the vertebral body when the balloon is in the uninflated configuration, the balloon configured to be positioned within the vertebral body such that the first section substantially opposes the first endplate and the third section substantially opposes the second endplate.

4. The balloon catheter of claim 3 wherein inflation of the balloon to the inflated configuration is configured to expand the band to an expanded configuration in generally a lateral direction relative to the vertebral body and form a cavity within the vertebral body for receiving a bone-filling material.

5. The balloon catheter of claim 4 wherein the band is configured to at least partially maintain the expanded configuration as the balloon is deflated to the uninflated configuration and as the shaft and the balloon are removed from the cavity formed within the vertebral body, wherein the band is configured to prevent leakage of the bone-filling material in generally the lateral direction relative to the vertebral body and facilitate adhesion of the bone-filling material to the vertebral body in generally a vertical direction relative to the vertebral body.

6. The balloon catheter of claim 1 wherein the section of the balloon around which the band is wrapped is substantially parallel to the longitudinal axis.

7. The balloon catheter of claim 1 wherein the section of the balloon around which the band is wrapped is aparallel to the longitudinal axis.

8. The balloon catheter of claim 1 wherein the band comprises an extracellular matrix (ECM) material.

9. The balloon catheter of claim 8 wherein the extracellular matrix (ECM) material includes small intestinal submucosa (SIS).

10. The balloon catheter of claim 1 wherein the band includes at least one of polylactic acid, polyglycolic acid, poly (ortho esters), poly (glycolide-co-trimethylene carbonate), poly-L-lactide-co-6-coprolactone, polyanhydrides, poly-n-dioxanone, poly (PHB-hydroxyvaleric) acid, surgical mesh, graft fabric, and mixtures and composites thereof.

11. The balloon catheter of claim 1 further including an adhesive for bonding the band to the exterior of the balloon in the uninflated configuration, wherein inflation of the balloon to the inflated configuration is configured to expand the band to an expanded configuration and break the bonding between the band and the balloon, wherein the band is configured to at least partially maintain the expanded configuration when the balloon is deflated to the uninflated configuration.

12. The balloon catheter of claim 1 wherein the band includes at least one slit configured to enhance expandability of the band.

13. The balloon catheter of claim 1 wherein the band has a substantially uniform height.

14. The balloon catheter of claim 1 wherein the band has a non-uniform height.

15. The balloon catheter of claim 1 wherein the distal end of the shaft pierces through the balloon along the section of the balloon around which the band is wrapped such that the shaft pierces through a proximal portion of the band, the proximal portion of the balloon, the distal portion of the balloon, and a distal portion of the band.

16. A method of performing kyphoplasty, the method comprising:
positioning a balloon catheter for insertion into a vertebral body having first and second endplates, the balloon catheter having a shaft including a proximal end, a distal end, and a lumen extending therethrough along a longitudinal axis, an inflatable balloon having an uninflated configuration and an inflated configuration disposed near the distal end of the shaft, the balloon having a proximal portion and a distal portion and an interior and an exterior, the interior being in fluid communication with the lumen, and an expandable band wrapped around a section of the exterior of the balloon in a generally proximal-distal direction;
inserting the balloon catheter with the balloon in the uninflated configuration into the vertebral body;
forming a cavity within the vertebral body including:
inflating the balloon to the inflated configuration and thereby expanding the band to an expanded configuration;
removing the balloon catheter from the vertebral body including:
deflating the balloon to the uninflated configuration and removing the shaft and the balloon from the vertebral body, wherein the band is configured to at least partially maintain the expanded configuration during deflation of the balloon, and wherein the band remains within the vertebral body during removal of the shaft and the balloon; and
dispensing a bone-filling material into the cavity, wherein the band is configured to prevent leakage of the bone-filling material in a generally lateral direction relative to the vertebral body and facilitate adhesion of the bone-filling material to the vertebral body in a generally vertical direction relative to the vertebral body.

17. The method of claim 16 wherein the balloon includes a first section, a second section, and a third section, the second section disposed between the first section and the third section, wherein the band is wrapped around the second section, wherein positioning the balloon catheter for insertion into the vertebral body includes positioning the balloon such that the first section substantially opposes the first endplate and the third section substantially opposes the second endplate such that the band is configured to expand to the expanded configuration in a generally lateral direction relative to the vertebral body.

18. The method of claim 16 wherein the section of the balloon around which the band is wrapped is substantially parallel to the longitudinal axis.

19. The method of claim 16 wherein the section of the balloon around which the band is wrapped is aparallel to the longitudinal axis.

20. The method of claim 16 wherein the band comprises an extracellular matrix (ECM) material.
